(19) United States
(12) Patent Application Publication
(10) Pub. No.: US 2008/0089905 A1
(43) Pub. Date: Apr. 17, 2008

(54) COMPOUNDS FOR STIMULATING STEM CELL PROLIFERATION INCLUDING AFA-OMEGA (EtOH)

(76) Inventors: Cyndy Davis Sanberg, Spring Hill, FL (US); Paula C. Bickford, Ruskin, FL (US); Paul R. Sanberg, Spring Hill, FL (US); Jun Tan, Tampa, FL (US); R. Douglas Shytle, Lutz, FL (US); Jerry Anderson, Klamath Falls, OR (US)

Correspondence Address: CHRISTOPHER PARADIES, PH.D., FOWLER WHITE BOGGS BANKER, P.A., 501 E KENNEDY BLVD, STE. 1900, TAMPA, FL, 33602, US

(21) Appl. No.: 11/757,251

(22) Filed: Jun. 1, 2007

Related U.S. Application Data
(63) Continuation-in-part of application No. 11/415,907, filed on May 2, 2006.
(60) Provisional application No. 60/676,733, filed on May 2, 2005.

(51) Int. Cl. A61K 36/05; A61K 36/45; A61K 36/82; A61P 43/00

(52) U.S. Cl. 424/195.17; 424/729; 424/732

(57) ABSTRACT

A method and composition for stimulating the proliferation and differentiation of stem cells is used to self-repair injury in mammals. A supplement is administered having an effective dose of substances selected from blueberry, carnosine, catechin, green tea extract, VitaBlue, Vitamin D3, Spirulina, AFA or effective combinations and derivatives of these. For example, a supplement comprising wild blueberry, green tea extract, carnosine, vitamin D3, and AFA-Omega (EtOH) or Spirulina and AFA-Omega (EtOH) exhibits a synergistic and unexpected association with substantially increased proliferation of bone marrow stem cells and CD34+. A therapeutic amount of a substance that is associated with a substantial increase in stem cells may be used to prevent and repair damaged tissues of the brain, due to stroke, the heart, due to coronary artery disease or a heart attack.

### RELATED APPLICATIONS

The present application is a continuation-in-part application of U.S. patent application Ser. No. 11/415,907, filed on May 2, 2006, claiming priority to No. 60/676,733 filed May 2, 2005 to Sanberg, et al., the disclosures of which are hereby incorporated herein by reference.

### FIELD OF THE INVENTION

The field of the invention is supplements for stimulating stem cell proliferation.

### BACKGROUND OF THE INVENTION

According to the online encyclopedia Wikipedia, Spirulina is a common name for human and animal food supplements produced primarily from two species of cyanobacteria: Arthrospira platensis, and Arthrospira maxima. These and other Arthrospira species were once classified in the genus Spirulina. There is now agreement that they are distinct genera, and that the food species belong to Arthrospira. The food supplement known as Spirulina is cultivated around the world, and is used as a human dietary supplement, available in tablet, flake, and powder form. While neither endorsing or refuting the dietary benefits of Spirulina, Wikipedia presents some support for some benefits of Spirulina as a dietary supplement, while also acknowledging that many of the claimed benefits of Spirulina are not supported by clinical research or accepted by the scientific community. Nevertheless, Spirulina has been used as a source of nutrition for at least hundreds of years, referencing the Aztecs, for example.

The Wellness Guide to Dietary Supplements, published by the University of California, Berkeley, teaches that blue-green algae is of little use as a food supplement. According to this guide, there are two main blue-green algae types used in dietary supplements: Spirulina and Aphanizomenon flos-aquae (AFA). AFA is chiefly harvested from Upper Klamath Lake in southern Oregon and then freeze-dried and sold in capsules and other forms. According to the guide, while blue-green algae contains small amounts of protein, vitamins (including C, E, and folate), beta carotene, and some minerals, a person would have to “ . . . eat huge amounts of algae,” to see any significant benefit, because they are a negligible source of nutrients. It also teaches that chlorophyll is of no use to the human body. It is thought that this is generally the scientific consensus, although promoters and advocates of blue-green algae disagree. The Wellness Guide recites:
Blue-green algae is not a medicine or a good source of nutrients. The few nutrients in blue-green algae are more plentiful and cheaper in foods. There's no scientific evidence that blue-green algae can treat or cure any illness or has any health benefit. In a recent court decision in California, marketers of blue-green algae were told to stop making health claims. Blue-green algae, especially AFA harvested from natural lakes, are easily contaminated with toxins such as microcystins and heavy metals. Because Health Canada, the equivalent of the U.S. FDA, found that some blue-green algae supplements (but not spirulina) contain high levels of microcystin, it has warned consumers—especially those thinking of giving AFA to their children—about potential contamination.
   We take a more balanced approach, acknowledging that some research indicates that replacing a very significant fraction of ingested calories by Spirulina and/or AFA might have beneficial effects on health, but also acknowledging that the amounts required to produce any measurable effect over a placebo is impractical to ingest in a normal diet or for an extended period.

Stem cells are found in many organs of the adult human including bone marrow, peripheral blood, umbilical cord blood, spleen, tooth pulp, and brain. These progenitor cells are being investigated for their potential use as transplanted tissues in the treatment of diseases such as cancer, diabetes, stroke, amyotrophic lateral sclerosis (ALS) and Parkinson's disease. Little effort however is being directed toward enhancing the endogenous stem cells in the adult as an avenue to promote healing. In many of these diseases, and in aging, stem cells and progenitors are known to have a reduced proliferative activity. For example, neural stem cells, muscle satellite cells, and endothelial progenitors all show reduced proliferation in the aged and may play a role in pathology of age-associated diseases (Kuhn et al., 1996; Conboy et al., 2005; Dimmeler and Vasa-Nicotera, 2003). In cardiovascular disease, for example, there is a correlation between a reduction in peripheral blood endothelial progenitor cells and many risk factors for cardiovascular disease (Vasa et al., 2001; Hill et al., 2003). As many of the diseases being targeted by stem cell therapies are age-associated diseases, selecting nutritional strategies that increase stem cell proliferation in the aged population seems appropriate.

Hematopoietic stem cells (HSCs) have been investigated for many years for their utility in cancer treatments. Experimental investigations of hematopoiesis and clinical approaches to correcting its deficiencies have focused on cytokine activity. Cytokines modulate hematopoiesis by maintaining the self-renewal of stem cells and stimulating the proliferation and maturation of committed progenitor cells required for the continuous replacement of mature blood cells (Ogawa 1993; Socolovsky et al. 1998; Whetton and Spooncer 1998).

In vitro, various combinations of cytokines including interleukin-1 (IL-1), IL-3, IL-6, stem cell factor (SCF), and erythropoietin (EPO) have been found to support the growth of multipotent progenitor cells (Henschler et al. 1994; Miller and Eaves 1997). Individually, granulocyte-colony-stimulating factor (G-CSF) and EPO are growth factors for committed myeloid and erythroid progenitors, respectively (Demetri and Griffin 1991). Clinically, G-CSF and EPO provide effective treatments for neutropenia and anemia (Adamson and Eschbach 1990; Eschbach et al. 1990) and are used to enhance peripheral blood progenitors as an alternative to bone marrow transplantation for cancer patients. However, such treatments are costly, and are not without certain risks.

Decreases in hematopoietic and endothelial progenitors are associated with aging. Decreases in certain hematopoietic progenitors has been reported in frail aging women (Semba et al., 2005). Endothelial progenitor cells (EPC) are also derived from bone marrow and found in the circulating blood. Circulating EPC's home to sites of neovascularization and injury (Penn et al., 2004) and can then differentiate into mature endothelial cells (Asahara et al., 1999). Declines in EPC's are noted in patients with coronary artery disease (Vasa et al., 2001), and when isolated from patients with high risk factors for coronary artery disease, show increased senescence in vitro (Hill et al., 2003). It has been suggested that endothelial progenitors play a role in cardiovascular homeostasis and that the decline observed in aging and disease tips the balance toward injury rather than repair. Exercise has been shown to increase EPC's and this may be one of the reasons that exercise has beneficial effects on cardiovascular disease (Laufs et al., 2004). Developing nutritional based strategies to increase progenitors could push the balance back towards repair, thus having a significant impact on health.

Neural stem cells also decline with aging (Kuhn et al., 1996) and some have postulated that declines in neurogenesis with aging are related to cognitive decline while others disagree (Bizon et al., 2004; Drapeau et al., 2003; Prickaerts et al., 2004). Nonetheless, it has been shown that nutritional treatments, such as feeding with blueberry, which improve cognitive function (Joseph et al., 1999) also increase neurogenesis (Casadesus et al., 2004). Thus, there is a correlation between improved neural stem cell proliferation and improved cognitive function.

While potentially better treatments are currently in development, few research studies have investigated the effects of natural products, vitamins, and other nutrients which may modulate self-renewal of stem cells. However, in recent years there has been an upsurge of interest on the effects of various dietary insufficiencies on hematopoietic and immune responsiveness. Folate, vitamin 1312, and iron have crucial roles in erythropoiesis. Erythroblasts require folate and vitamin B12 for proliferation during their differentiation. Deficiency of folate or vitamin B12 inhibits purine and thymidylate syntheses, impairs DNA synthesis, and causes erythroblast apoptosis, resulting in anemia from ineffective erythropoiesis (Koury and Ponka, 2004). Other studies have recently found that dietary fatty acids, particularly oleic acid and linolenic acid, actively promote the proliferation of hematopoietic stem cells (Hisha et al., 1997; Hisha et al., 2002) as well as modulate the self-renewal of intestinal epithelial cells (Holehouse et al., 1998). Vitamin D has also received increasing attention over the past few years, in part, because recent studies suggest that nearly half the US population may be vitamin D deficient (Meyer, 2004). Recent laboratory studies demonstrate that vitamin D3 has a dramatic effect on stimulating the proliferation of various forms of multipotent progenitor cells, particularly those involved with the immune system (Mathieu et al., 2004). Recent laboratory research on cellular senescence (the end of the life cycle of dividing cells) suggests that the dietary nutrient, carnosine, found in muscle and brain of mammals, has the remarkable ability to rejuvenate cells approaching senescence, restoring normal appearance and extending cellular life span (Hipkiss et al., 1998; Holliday and McFarland, 2000).

The use of fruits or vegetables has the benefit of providing a cocktail of many different phytochemicals with multiple actions including antioxidant and antiinflammatory effects and is one reason they have been extensively studied in the field of cancer biology. Other studies suggest dietary supplementation with foods high in antioxidants, such as blueberries, can prevent and even reverse cellular and behavioral parameters that decline as a function of aging (Joseph et al., 1999; Gemma et al., 2002). For example, dietary supplementation with 2% blueberry extract has produced both neuroprotective and neurorestorative effects in aged animals, perhaps as a result of modulation of cell signaling cascades (Williams, Spencer et al. 2004). Furthermore, blueberry extract has been shown to increase neurogenesis in the aged rat brain (Casadesus, NSci Abstract, 2002). We have shown that feeding blueberries to aged rats increases the survival and growth of hippocampal grafts grown in the anterior chamber of the eye (Willis et al., 2005), demonstrating that nutritional supplementation can not only increase proliferation of tissues, but promote appropriate differentiation.

Green tea is a drink made from the steamed and dried leaves of the Camellia sinensis plant, a shrub native to Asia. Green tea has been widely consumed in Japan, China, and other Asian nations to promote good health for at least 3,000 years. Recently, scientists have begun to study it's health effects in animal, laboratory, and observational human studies. Although active compounds within green tea extract have been shown to inhibit the growth of a number of tumor cell lines, they do not effect the growth of normal cells at similar concentrations (Chen et al., 1998; Wang and Bachrach, 2002) and actually may provide cellular protection from aging (Song et al., 2002).

In light of such findings reviewed above, it appears that certain nutrients, vitamins, and flavonoids could have important roles in maintaining the self-renewal of stem cells and stimulating the proliferation and differentiation of committed progenitors required for the continuous replacement of mature cells in the blood, brain, and other tissues. Furthermore, it may be possible to use certain natural products, either alone or synergistically, for the treatment of conditions where the stem cell replacement appears warranted such as aging or diseases associated with aging.

However, the amounts of such substances that have shown actual results in pre-clinical studies in mammals and in clinical studies in humans are impractical to implement as supplementation to an ordinary diet. Even if studies are correct about the value of these substances, consumption of sufficient quantities to substantially improve health is impractical. A very substantial portion of an adult human's caloric intake would need to be devoted solely to a specific food group, such as the dietary supplement Spirulina.

Aged mammals, such as rats, dogs and humans, can improve age-related declines in motor abnormalities and cognitive abnormalities with dietary interventions that include foods with a high antioxidant capacity. Antioxidants work at the cellular level; therefore, it would be expected that benefits of antioxidants in one mammal would be mirrored in other mammals. Certain foods were identified on the basis of the ability to show antioxidant activity in vivo in mammals and in an in vitro assay. Hundreds of foods were examined using this assay (Cao et al., 1997) and several were chosen with high in vitro antioxidant activity for testing in vivo. For example, when 18 month old rats are fed a diet in which 2% of the diet is a blueberry extract, after 2 months on this diet, we observe a significant improvement in motor performance on a balance beam (Joseph et al., 1999). We also observed a significant improvement on a Morris water maze in rats fed a diet supplemented with large quantities of strawberry, blueberry or spinach (Joseph et al., 1999). These same animals also show improved dopamine release in the striatum. A spinach diet improves age-induced deficits in motor learning using either a rod running motor learning task or classical eye blink conditioning (Bickford et al., 2000; Cartford et al., 2002). Markers of inflammation, such as the pro-inflammatory cytokine TNFα are increased in the brains of PD patients (Mogi et al., 1996), and 30 days following 6-OHDA lesions. We have shown that these diets decrease markers of oxidative damage and pro-inflammatory cytokines (Gemma et al., 2002; Cartford et al., 2002), furthermore these changes are related to the foods antioxidant activity as foods such as cucumber which are low in antioxidant activity have no effect (Gemma et al., 2002). We have been examining these diets in an animal model of Parkinson's disease. We have preliminary data showing that the blueberry or Spirulina diet will increase the immune response 7 days following an insult and then prevent the prolonged activation of microglia at later time points. It is this later prolonged activation which we hypothesize is detrimental and reflects the ongoing inflammation observed in Parkinson's disease.

An additional class of compounds have also been suggested to have activity in promoting stem cell function. The dietary supplement known as Spirulina is abundant in phycocyanin which gives spirulina a blue pigmentation. The large amount of chlorophyll accounts for the vivid green color. Other additional carotenoids present contribute to its rich pigmentation. These same compounds are thought to provide an antioxidant effect. Spirulina has been shown to increase adult neurogenesis following an inflammatory insult and in aging. (Vila et al, 2005; Vila et al., 2006)

Jensen et al., in U.S. Pat. No. 6,814,961, previously showed that high dosages, i.e., 5 g of dried AFA, a species of blue green algae, were able to increase the number of circulating CD34+ stem cells. However, consumption of water-soluble phycocyanin rich fraction of AFA did not alter the number of circulating CD34 stem cells. Moreover, consumption of a carbohydrate rich fraction of AFA, derived from mechanical separation from the water-soluble AFA actually decreased the number of circulating CD34 stem cells.

While benefits are known for incorporating antioxidants into the diet of humans, adjusting diets to incorporate a large proportion of these foods is difficult and often fails to incorporate sufficient amounts of antioxidants to make a significant difference on proliferation and differentiation of bone marrow cells, CD34⁺ HSCs, CD 133⁺ progenitor cells from peripheral blood or any other stem cells. It would be of great benefit to identify certain natural compounds that can promote proliferation of hematopoietic stem cells or other stem cells, synergistically, such that the natural compounds could be taken in the form of a supplement that would have a significant, measurable effect.

U.S. patent application Ser. No. 11/415,907, to Sanberg, et al., (having inventors in common with the inventors of the present invention), which was filed on May 2, 2006, claiming priority to No. 60/676,733, filed May 2, 2005, (referred to herein as “Sanberg et al.”) discloses compounds and methods of use of the compounds for practical stimulation of stem cell proliferation in animals, especially mammals, such as dogs, cats, murine and human. The experiments reported in the drawings and disclosure clearly show a synergistic improvement in effectiveness by combining specific natural ingredients in a supplement. According to Sanberg, et al., three promising natural substances were studied for their potential use as antioxidants, especially for protective effects against stroke in rats, blueberry, spinach and the dietary supplement known as Spirulina. Each, in sufficient quantities in a diet, have a significant, differential effect on reducing ischernia-induced caspase-3 activity and cerebral infarction. Animals were put on a diet of either control, blueberry (10,000 mg/kg/day), spinach (10,000 mg/kg/day) or the dietary supplement known as Spirulina (1500 mg/kg/day) for 4 weeks prior to the insult. We used a 60 minute occlusion of the middle cerebral artery and at 24 hours examined the size of the infarct using TIC staining. We found a 70% protection in infarct size in the spirulina treated rats and a 50% protection in both the blueberry and spinach treated rats. In these animals we have observed a significant decrease in caspase-3 activity and the number of TUNEL positive cells indicating that a reduction of apoptosis was achieved. All groups also showed significant improvement on horizontal and vertical activity measures when compared with controls. However, the amount of antioxidant consumed per day makes it difficult to sustain the benefits of any of these diet plans long term, which supports the general consensus that the value of these nutrients as dietary supplements is not supported.

Several whole food extracts, herbal extracts, and specific compounds were screened individually for proliferative activity on human bone marrow cells in culture at reasonable doses as nutritional supplements: spinach, the dietary supplement known as Spirulina, EGCG, epicatechin, withania, somnifera, carao, rehmania glutinosa, and astragulus membranaceous. These supplements did not show a substantial increase in activity on proliferation of human bone marrow stems cells in this in vitro study. Herein, we consider a substantial increase in activity of proliferation of stem cells to be at least ten percent (10%) improvement over a control. These compounds, which did not show a substantial improvement in this in vitro study were not further tested, because it was thought that they had little potential for success as supplements for stimulating stem cell proliferation.

Sanberg et al. disclosed synergistic, surprising and unexpected results when blueberry extract was combined with other nutritional supplements that showed some benefit in stimulating human bone marrow stem cell proliferation, as shown in FIG. 2 (also FIG. 2 of Sanberg et al.), for example. A reasonable supplement amount of a compound containing at least three substances from the group of substances consisting of blueberry, carnosine, catechin, green tea extract, and vitamin D₃ showed much greater effectiveness in stimulating stem cells of human bone marrow, CD34+, and CD133+ than the equivalent dose of a supplement administered without compounding with any of the other substances from the group. While Granulocyte-macrophage colony stimulating factor, human (hGM-CSF) is well known for its effectiveness in stimulating proliferation of stem cells, it is limited to use for life sciences research, and cannot be used as a dietary supplement. FIG. 2 shows that the synergistic effects for combinations of blueberry, carnosine, catechin, green tea extract, and vitamin D₃ caused greater in vitro stimulation of stem cells than hGM-CSF, which is very surprising and unexpected. In vivo studies provided clear evidence that the results obtained in vitro applied in practical applications in vivo, as well.

### BRIEF SUMMARY OF THE INVENTION

Claims of a promoters and advocates of health benefit attributable to AFA supplements led the investigators to test AFA supplements for proliferative effects on stem cells. FIG. 3 shows that a type of AFA referred to herein as AFA-Alpha showed the most promise for substantial increases in proliferation of bone marrow stem cells compared to a control at reasonable doses. As shown in FIG. 4A, for example, a blue-green algae of AFA identified as Alpha is shown to produce a substantial increase (i.e. greater than 10%) in proliferation of bone marrow stem cells. The AFA-Alpha supplement, whether processed using water or ethanol, increased proliferation substantially more than the AFA-Omega supplement. This promising screening caused the investigators to consider whether combining AFA-Alpha with a compound containing blueberries, green tea extract, carnosine, catechin and/or vitamin D₃ might yield a synergistic effect. Thus, the AFA-Alpha supplement was tested in combination with the compound containing blueberry extract, carnosine, green tea extract and vitamin D₃ that showed an unexpected synergistic effect in Sanberg et al., as shown in FIG. 2 (Composition A). Composition A includes 500 ng/ml blueberry, 20 μM carnosine, 500 ng/ml green tea, and 5 μM of vitamin D₃.

As shown in FIG. 4B, AFA-Alpha (labeled Alpha H20 and Alpha EtOH) failed to substantially increase (i.e. at least 10%) proliferation of bone marrow stem cells compared to the increase caused by Composition A, alone, without any addition of a blue green algae. In this case, an additive effect would have been expected to increase stem cell proliferation by about 20% or more over control. Thus, the test using AFA-Alpha not only failed to show any synergy, but also failed to produce any substantial increase that would be expected if AFA-Alpha merely produced an additive effect. These tests with AFA-Alpha teach away from combination of AFA with Composition A.

Tests of AFA-Omega, either hydrated in water (H20) or processed with ethanol (EtOH) yielded a surprising and unexpected result. The AFA-Omega hydrated in water resulted in no substantial increase in bone marrow stem cell proliferation when combined with Composition A, as shown in FIG. 4B, and its effect on proliferation was less than that of AFA-Alpha, as it would be expected from the lesser effect on proliferation observed in the screening test of FIG. 4A (labeled Omega 62.5, Omega 125, Omega 250 and Omega 500). However, for reasons that are still not understood, the AFA-Omega processed in ethanol, while it did not show any difference in the screening test of FIG. 4a when compared with AFA-Omega hydrated with water, it did exhibit a substantial increase (i.e. at least 10%) in bone marrow stem cell proliferation compared to Composition A without the addition of AFA-Omega (EtOH-processed). There is no reason to select AFA-Omega and the ethanol processing route and no reason to believe that there should be any reasonable expectation of success using this variety and processing route, considering the utter failure of all of the other AFA compounds tested to add substantially to the normal increase in stem cell proliferation of Composition A.

An advantage of a compound made of Composition A and AFA-Omega (EtOH) is that the proliferation of stem cells may be substantially increased compared to the use of Composition A.

The investigators tested a Spirulina, which was previously screened out due to its poor performance in a screening trial as discussed in Sanberg et al., In FIG. 1A, Spirulina at a high dose is shown to increase bone marrow stem cell proliferation by at least ten percent (i.e. a substantial increase). In an investigation of the effect of Spirulina on CD34+, a dose one-half of the maximum dose tested achieves nearly the same substantial increase in cell proliferation as the higher dose. The data shows that lower doses do not substantially increase CD34+ proliferation. In combination with Composition A, the higher dose of Spirulina nearly does not substantially increase cell proliferation compared to Composition A (labeled NT020). However, for bone marrow stem cells, the high dose of Spirulina not only substantially increases the proliferation compared to Composition A, alone, but also the combination of Composition A and Spirulina may synergistically increase proliferation, yielding an increase in stem cell proliferation greater than the additive additions of the individual effects of Composition A and Spirulina. This is a very surprising and unexpected result, as blue-green algae generally fails to produce any substantial increase in proliferation in combination with Composition A compared to Composition A, alone.

Certain natural products, when combined, exert a synergistic proliferation of human bone marrow cells, CD34+, and/or CD133+ progenitors. A method of increasing stem cell proliferation and, in some cases, selective migration, and compositions have been found that synergistically increase the proliferation compared to individual foods or supplements, such that a combination of these natural substances shows a substantial increase in proliferation by administering small quantities of specific natural substances in mammals.

A compound for increasing stem cell proliferation includes blueberry, a Spirulina and/or AFA-Gamma (EtOH), and a substance selected from the group of substances consisting of carnosine, catechin, green tea extract, and vitamin D₃. By selecting one or more substances from the group of substances and compounding the substances into a supplement that is digestible in the digestive tract, a compound may be formed that exhibits synergistic proliferation of stem cells compared to any of the substances taken alone. Administering the compound in an effective dose to a mammal is shown to synergistically increase measurable indicators of proliferation of certain stem cells, to protect against damage and/or to repair damage. A method includes selecting a compound having blueberry, Spirulina and a substance selected from the group of substances consisting of carnosine, catechin, green tea extract, and vitamin D₃, selecting a dose for stimulating stem cell proliferation in an animal, such as by measuring blood levels of one or more of the substances in the compound, and administering the dose at least daily to achieve a substantial increase in stem cell proliferation.

### BRIEF DESCRIPTION OF DRAWINGS

The following drawings and examples are examples of the present invention only and are not to be read as limiting issued claims or any limitations of the issued claims.

FIGS. 1A and 1B show graphical data for (A) in vitro screening of a Spirulina and (B) a synergistic increase in stem cell proliferation of the combination of Spirulina and Composition A (labeled NT020).

FIG. 2 shows synergistic effects of combinations of natural ingredients, including a combination of natural ingredients included in Composition A (labeled BB/D3/GT/Ca) from Sanberg et al.

FIGS. 3A and 3B illustrate graphical data for a test similar to the test in FIGS. 1A and 1B, except no synergistic effect is seen for CD34+ proliferation by the combination of Composition A and Spirulina.

FIGS. 4A and 4B illustrate graphical data for a test similar to the test in FIGS. 1A and 1B, except no synergistic effect is seen for proliferation of bone marrow stem cells by the combination of Composition A and various versions of AFA, but a surprising and unexpected increase in stem cell proliferation is shown for an AFA-Omega (labeled in 4A as Omega (E); and labeled in 4B as Omega EtOh) processed using ethanol, when combined with Composition A, when compared to combinations of Composition A and either AFA-Alpha (water suspension or EtOH-processed) or AFA-Omega water suspension.

FIGS. 5A and 5B illustrate a comparison similar to the comparison in FIGS. 4A and 4B, except data shown is for (A) CD34+ proliferation using two versions of AFA (water suspensions only) and (B) Composition A (labeled NT020) and combinations of Composition A and AFA-Alpha (labeled w/Alpha 500), AFA-Omega (labeled w/Omega 500), AFA-Alfa using ethanol process (labeled w/Alpha (E) 500), and AFA-Omega using ethanol process (labeled w/Omega (E) 500, showing substantially increased proliferation over Composition A, alone, only in combination with AFA-Omega using ethanol process.

### DETAILED DISCLOSURE

Three antioxidants were compared for protective effects against stroke in rats: blueberry, spinach and spirulina. Results show that each, in sufficient quantities in a diet, have a significant, differential effect on reducing ischemia-induced caspase-3 activity and cerebral infarction. Animals were put on a diet of either control, blueberry (10,000 mg/kg/day), spinach (10,000 mg/kg/day) or spirulina (1500 mg/kg/day) for 4 weeks prior to the insult.

After a 60 minute occlusion of the middle cerebral artery, the size of the infarct was examined using TTC staining at 24 hours. A 70% protection in infarct size in the spirulina treated rats and a 50% protection in both the blueberry and spinach treated rats is measured. In these animals, a significant decrease in caspase-3 activity and the number of TUNEL positive cells indicates that a reduction of apoptosis is achieved, when one of the three antioxidants is administered. All groups also showed significant improvement on horizontal and vertical activity measures when compared with controls. However, the amount of antioxidant consumed per day makes it difficult to sustain the benefits of any of these diet plans long term.

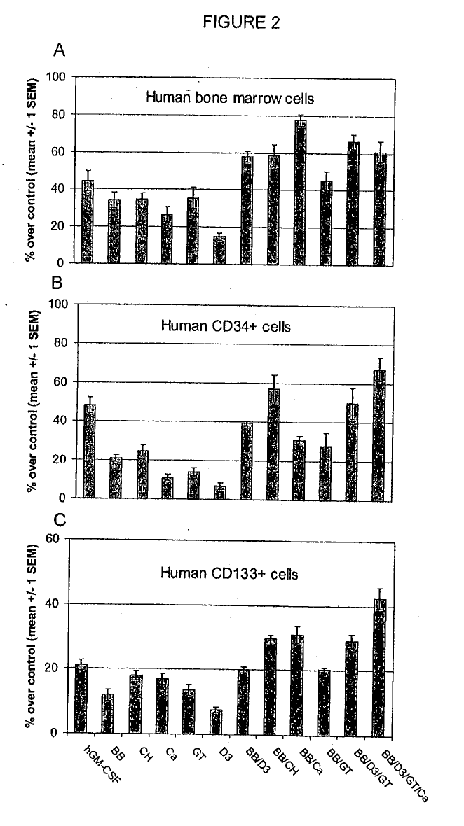

FIGS. 2(A-C) show synergy of a combination of Blueberry extract with D3, CH, D3/GT or D3/GT/Ca in producing proliferation of stem cells compared to a control. In FIG. 2(A), human bone marrow cells are cultured in 96-well tissue-culture plates (5×10⁴/well) and treated with blueberry extract (500 ng/mL) in the presence of D3 (5 μM), CH (20 μM), Ca (20 4 M), GT (500 ng/mL), D3 (5 μM)/GT (500 ng/mL) or D3 (5 μM)/GT (500 ng/mL/Ca (20 μM) for 72 hours. Human bone marrow-derived CD34+ cells (5×10⁴/well) are treated as per FIG. 2(A) in FIG. 2(B). For MTT assay, these cells were prepared for cell proliferation analysis. Data were also represented as the percentage over control. ANOVA and post hoc testing shows significant differences of mean percentage over control (+/−SD with n=3 independent experiment) between individual and certain combined treatments, for A, BB/D3 combined treatment compared to BB or D3 individual treatment (p<0.005), BB/CH compared to BB or CH (p<0.005), BB/Ca compared to BB or Ca (p<0.001), BB/D3/GT compared to BB, D3 or GT, BB/D3/GT/Ca compared to BB, D3, GT or Ca; for B, BB/CH combined treatment compared to BB or CH individual treatment (p<0.005), BB/D3/GT/Ca compared to BB, D3, GT or Ca (p<0.001). Human CD133+ cells were cultured for 23 days in the presence or absence of green tea (50 μg/ml). Cells were then fixed and stained for A2B5 Left panels (RED), NF200 middle panels (Green) or TUJ1 right panel (red). Nuclei were stained with DAPI (Blue). In both conditions there were some cells that were positive for all markers which is consistent with literature that has shown that CD133+ cells can differentiate into neural lineages. In the cells treated with green tea, the staining was significantly more prevalent.

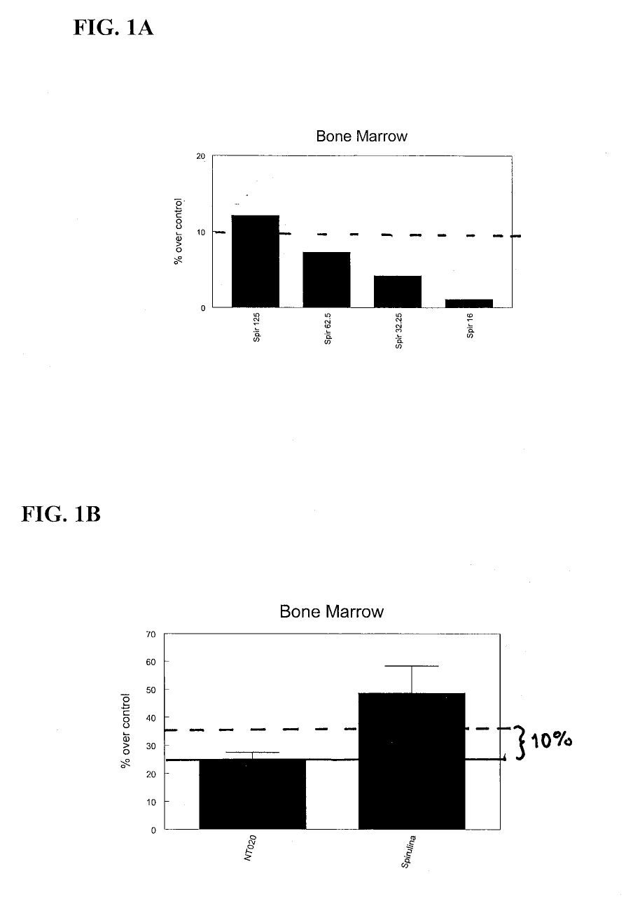

FIGS. 1A, 3A, 4A and 5A show that, on their own, blue-green algae increases cell proliferation of stem cells in a dose-dependent manner. This is consistent with results of Sanberg et al. Cell proliferation, as determined by MTT assay, is displayed as the percent of cell proliferation over the control, which represents cells cultured in the same condition without any extract or compound added.

A combination of extracts and compounds results in a greater percentage of proliferation than observed with the individual extracts and compounds for those compounds shown in FIG. 2(A-C). For example, blueberry/vitamin D3 exhibited a 62% increase in proliferation, blueberry/catechin a 70% increase, and blueberry/carnosine with the greatest synergistic affect of 83% (FIG. 2 A). Blueberry/green tea, blueberry/vitamin D3/green tea, and blueberry/vitamin D3/green tea/carnosine also displayed significant increases in proliferation of 56%, 72%, and 70%, respectively, in FIG. 2A and significant synergistic effects are shown in FIGS. 2B and 2C, also.

Reagents. All compounds were added to cell cultures as described in the results sections. Sources of compounds may be as follows: blueberry (freeze dried powder, Van Drunen Farms, Momence, Ill.), green tea extract (Rexall), Carnosine (Sigma), Catechin (Sigma), and the activated form of vitamin D3 (25-Hydroxycholecalciferol, Sigma), for example.

Cell cultures and MTT Assay. For cell proliferation analysis, human bone marrow cells, human CD34⁺ cells or CD133 cells (BioWhittaker, Inc.) were cultured in 96 well plates (5×10⁴/well) containing 100 μL of complete medium (RPMI 1640 medium supplemented with 5% fetal calf serum). These cells were treated for 72 hours with various extracts at a wide range of doses (8 ng/mL to 500 ng/mL) or molecular compounds (0.3125 μM to 20 μM). Five hours before the end of the treatment, 20 μL of MTT solution (MTT kit, Sigma) was added to each well. These plates were then incubated in a CO₂ incubator at 37° C. for 5 hours and the cultured media removed with needle and syringe. 200 μL of DMSO was added to each well with pipetting up and down to dissolve crystals. These plates were put back into the 37° C. incubator for 5 minutes, transferred to plate reader and measured absorbance at 550 nM. Data were represented as relative percentage mean proliferation, defined as O.D. reading number of each treated cells normalized to control cells (in the absence of treatment).

Promotion of Bone Marrow Cell Proliferation in a Dose-dependent Manner. Certain whole food extracts, such as Blueberry (BB), Green Tea (GT), and specific compounds, including Catechin (CH), Carnosine (Ca), and Vitamin D3 (D3), were found to increase cell proliferation of human bone marrow cells in a dose dependent manner (FIG. 1). Cell proliferation as determined by MTT assay is displayed as the percent of cell proliferation over the control, which represents cells cultured in the same condition without any extract or compound added. The positive control, human granulocyte colony-stimulating factor (hGM-CSF; FIG. 1A), produced a 44.5±8.1% proliferation at the highest dose of 100 ng/mL. Blueberry (BB) and CH demonstrated a 34.5±6.7 and 34.8±5.2% increase in proliferation at 500 ng/mL and 20 μM respectively (FIGS. 1B, 1C). The compound, Ca displayed a 26.6±6.0 increase at 20 μM (FIG. 1D), and D3 displayed a lower percentage of proliferation, 14.8±3.3% at 5 μM (FIG. 1F). Green tea (GT) produced a proliferation similar to BB and CH with 35.6±9.2% proliferation at 500 ng/mL (FIG. 1E).

Synergistic Stimulatory Effect of Extracts and Compounds on Proliferation. Cultured human bone marrow cells were cultured with different varieties of blue-green algae, extracts and compounds and combinations of these. A positive control, hGM-CSF displayed 48.3±7.4% proliferation, while BB, CH, Ca, GT, and D3 alone did not cause proliferation in a significantly different manner as demonstrated in FIG. 2A. However, the combination of extracts and compounds resulted in a greater percentage of proliferation than observed with the individual extracts and compounds. For example, BB/D3 exhibited a 62% increase in proliferation, BB/CH a 70% increase, and BB/Ca with the greatest synergistic affect of 83% (FIG. 2A). BB/GT, BB/D3/GT, and BB/D3/GT/Ca also displayed significant increases in proliferation of 56%, 72%, and 70% respectively (FIG. 2A).

Promotion of CD34⁺ Cell Proliferation and Synergistic Properties of Extracts and Compounds. To determine whether these extracts and compounds promoted cell proliferation of other progenitor cells, we cultured CD34⁺ human hematopoietic stem cells under the same conditions as the bone marrow cells using different combinations of the extracts and compounds, and with the individual extracts and compounds at the highest doses determined to promote the greatest amount of proliferation in the bone marrow cell studies, which was represented by FIG. 1A-F. The results revealed a 48.3±7.4 increase for hGM-CSF, which was approximately a 5% increase in proliferation as compared to hGM-CSF effect on the bone marrow cells (FIG. 2B). However, individually, BB, CH, Ca, GT, and D3 displayed a 20.9±3.0, 24.8±5.0, 11.05±2.1, 14.0±3.7 and 6.9±2.6 increase in proliferation respectively, which are much lower than observed in the bone marrow cells (FIG. 2B). However when combined, BB/D3, BB/CH, BB/Ca, BB/GT, and BB/D3/GT demonstrated a 39.3±2.0%, 57.3±10.4%, 30.9±3.4%, 27.9±10.0%, and 49.9±13.1% increase in proliferation respectively which is at least additive and in some synergistic (FIG. 2B). Interestingly, the combination of BB/D3/GT/Ca resulted in an increase of 67.6±11.9%, a simple additive effect would have been 52% demonstrating a synergistic effect of the combination (FIG. 2B).

Promotion of CD133+ Cell Proliferation and Synergistic Properties of Extracts and Compounds. Some of the compounds and combinations with the greatest activity on proliferation of the bone marrow derived CD34+ cells were then used to treat CD133+ (progenitor cells) collected from peripheral blood and cultured under the same conditions as above. Cell proliferation was determined by MTT Assay and is displayed as the percent of cell proliferation over the control. The results revealed an 21.11±2.9% increase after treatment with hGM-CSF (FIG. 2C). Individually, BB, Ca, GT, and D3 displayed a 11.9±3.1, 16.9±3.3, 13.57±3.0, 7.6±1.39% increase in proliferation respectively (FIG. 2C). When combined, BB/D3/GT, and BB/D3/GT/Ca demonstrated a 29.2±3.6 and 42.5±5.9% increases in proliferation (FIG. 2C) of human CD133⁺ cells.

The results of in vivo studies in Sanberg et al. demonstrate that various natural compounds and their combinations promote the proliferation of human bone marrow, human bone marrow derived CD34⁺, and human peripheral blood derived CD133⁺ cells. When tested individually, these compounds are most effective in promoting proliferation of the bone marrow cells and less effective when used to treat the progenitor populations. This finding may reflect an effect of the individual compounds on the mature cell populations that are also present in the bone marrow cell cultures. When the activity of the compounds was examined in combinations, the additive and synergistic effects were more profound in the progenitor CD34⁺ and CD133⁺ cells. AFA-OMEGA (EtOH) or Spirulina, on the other hand, tended to increase proliferation of bone marrow stem cells more than progenitor cells and to increase proliferation of bone marrow stem cells more than progenitor cells even when combined with Composition A.

It is likely that an oral formulation of Spirulina or AFA-Omega (EtOH) with blueberry extract, green tea extract, carnosine, and vitamin D3 may be used as a dietary supplement to promote healing naturally in various parts of the body where progenitor cells are in need, such as in the case with various disease states or with an injury.

Composition B for the human formulation comprised Green tea of 5.7 mg/kg, Carnosine of 1.4 mg/kg, Vitamin D3 of 0.71 μg/kg, Blueberry=5.1 mg/kg, and Vitablue=0.6 mg/kg. The composition for the formulation needed to produce a similar blood level in mice was administered at 10 times the human formulation (Composition C). Significant protection is provided at both doses compared to the control group. The human equivalent dose shows a marked protection against oxidative damage.

An example of a supplement formulation may include 360 mg of blueberry. Alternatively, an equivalent amount of VitaBlue may be added. It is thought that anthocyanins are the active ingredient in blueberries, and VitaBlue is enriched by ten times in anthocyanins compared to blueberry. Thus, it is believed that 40 mg of VitaBlue is equivalent to 400 mg of blueberry. A pharmacokinetic study (pK study) in humans [Mazza G, Kay C D, Cottrell T, Holub B J (2002) Absorption of Anthocyanins from Blueberries and Serum Antioxidant Status in Human Subjects J. Agric. Food Chem., 50, 7731-7737](7) found consumption of 1200 mg blueberry anthocyanins resulted in human plasma conc. of 17 ng/ml. The equivalent of 800 mg of blueberry (1.2% anthocyanins) would lead to a blood concentration of approximately 0.14 ng/ml anthocyanins by interpolation. The doses tested in vitro ranged from 0.08 to 5 ng/ml anthocyanins. In humans, this range corresponds to a dose in a range from 400 mg to 25 grams of blueberries and/or their equivalent. The upper range has been tested as a supplement in our tests for aging and other studies. In practical terms, 25 grams of blueberry would be about 12 pills, which is impractical for supplements provided in pill form. More preferably, the upper limit for blueberry and equivalents is about 5 grams per day. Even more preferably, blueberry is compounded with other supplements that provide a synergistic effect.

Our in vitro data for activity of green tea extract tested a range from 4 ng/ml to 250 ng/ml catechins (assuming only 10% of the catechins went into solution, we obtain a range of 0.4-25 ng/ml). A pK study in humans [Manach C, Gary Williamson, Christine Morand, Augustin Scalbert, and Christian Rémésy (2005) Bioavailability and bioefficacy of polyphenols in humans. I. Review of 97 bioavailability studies. Am J Clin Nutr; 81 (suppl):230S-42S](6) found that consumption of 500 mg catechins resulted in a plasma conc of 2 nmol/l. Using the MW of catechin at 280, 2 nmol/l=0.58 ng/ml. Thus, in humans 400 mg GTE will result in a 0.4 ng/ml plasma concentration, and a preferred range of green tea extract is from 400 mg to 25 grams. For practical considerations, the amount of green tea extract is selected to be no greater than 5 grams. More preferably, green tea extract is compounded with other substances to provide a synergistic effect.

In one example, Vitamin D3 is used as 2000 IU's, which is equivalent to 50 μg. In humans, daily administration of 4000 IUs (100 mcg) results in a blood concentration of 100 nmol/L. There is no official RDA for vitamin D3 [R. Vieth, D. Fraser, Vitamin D insufficiency: no recommended dietary allowance exists for this nutrient, CMAJ 166 (2002) 1541-1542](4). According to a conservative report of the Food and Nutrition Board [R. Vieth (2004) Why the optimal requirement for Vitamin D3 is probably much higher than what is officially recommended for adults. Journal of Steroid Biochemistry & Molecular Biology 89-90 (2004) 575-579], the safety limit (no adverse effect level, NOAEL) of vitamin D3 intake in humans is 60 mcg (or 2400 IU) per day [Standing Committee on the Scientific Evaluation of Dietary Reference Intakes. Dietary Reference Intakes: Calcium, Phosphorus, Magnesium, Vitamin D, and Fluoride. National Academy Press, 1997]. After applying a margin of safety, the recommended upper limit is 50 mcg (2000 IUs) per day for intake by the general public [I. Munro, Derivation of tolerable upper intake levels of nutrients, Am. J. Clin. Nutr. 74 (2001) 865-867]. Clinical trials show no benefit from oral doses 20 mcg (800 IUs) or less [R. Vieth (2004) Why the optimal requirement for Vitamin D3 is probably much higher than what is officially recommended for adults. Journal of Steroid Biochemistry & Molecular Biology 89-90 (2004) 575-579](1). Recent, clinical trials also report that human oral doses of 1000 IUs (20 mcg/day) to 4000 IUs (100 mcg/day) are completely safe and within normal levels produced by exposure to the sun (total body sun exposure=10,000 IUs/day) [R. Vieth (2004) Why the optimal requirement for Vitamin D3 is probably much higher than what is officially recommended for adults. Journal of Steroid Biochemistry & Molecular Biology 89-90 (2004) 575-579](1). A dose of 50 mcg leads to a plasma concentration of 0.05 μM. Doses tested in vitro ranged from 0.07 to 5 mM, a range of 50 μg to 5000%1 g in humans, which is from 2000 IU to 20,000 IU. In practice, 20,000 IU's is beyond any limit recommended and might lead to hypercalcemia. In a preferred example, the range of Vitamin D3 is selected in a range from 2,000 to 4,000 IU.

In one example, carnosine is added to a supplement in a dose of 100 mg. A recent pharmacokinetic study in humans [Park Y J, Volpe S L, Decker, E A (2005) Quantitation of Carnosine in Humans Plasma after Dietary Consumption of Beef. J. Agric. Food Chem., 53, 4736-4739] found that two beef patties contain 250 mg carnosine and when ingested by humans resulted in a plasma conc. of 33 mg/l which peaked at 3 hrs and returned to baseline at 5 hrs. With a Mw of 266 and assuming 5 liters of blood in a human, 250 mg carnosine produces a blood concentration of 620 μM. Assuming linear pharmacokinetics, 100 mg should produce a human blood concentration of 248 μM. Doses tested ranged from 1-20 μM, effect was still increasing at 20 μM. Thus, a dose as high as 620 mg may be helpful and still be within normal ranges for human consumption. A preferred effective range is about 10 mg to 100 mg of carnosine based on extrapolation from results obtained.

In one preferred example, at least three substances are selected to be compounded into a supplement tablet, pill, or other form for ease of administering a dose. In humans, a ratio of blueberry to Vitamin D3 may be selected from 400:0.1 to 1:4000 or 25, 000:0.05 to 1:500,000, based on the previous maximum and minimum ranges for each. However, limiting the doses of each to 5 grams, yields ratios from 5, 000:0.5 to 1:100,000 for the highest to lowest ranges of each component.

Similarly, a ratio may be calculated for blueberry to green tea. Thus, the lowest to highest ratio is in a range from 4:50 to 0.08:1, and the highest to lowest ratio is in a range from 50:4 to 12.5:1.

In another example, several ratios may be selected for a combination of blueberry to carnosine to green tea extract to Vitamin D3. Examples would include from 4, 000:4,000:100:1, such as a composition having Vitamin D3 at the recommended highest level, or 100,000:100,000:2,000:1, with Vitamin D3 at its low level.

Two groups of adult male Sprague-Dawley rats initially received supplementation using Composition I (n=8) or vehicle (n=7). Composition 1 equals (blueberry 3 mg/kg/day; Vitamin D3 1 mg/kg/day; green tea 3 mg/kg/day; and carnosine 10 mg/kg/day. Dosing for Composition I and vehicle consisted of daily oral administration (using a gavage) over a two-week period. Results and procedures for testing in rats are disclosed in Sanberg et al., which is incorporated by reference herein in its entirety. The Composition A supplementation reduces the glial scar/ischemic area damage in the striatum compared to a control group (Vehicle). A significant decrease in mean glial scar area (e.g. 75%) was observed in the ischemic striatum of animals having Composition A supplementation compared to that of vehicle-treated animals. These histological results parallel the improved behavioral performance for Composition A supplementation found in the Bederson Test and EBST. The results that correlate Composition A supplementation to cell proliferation in vitro also occur in vivo. It is believed, without being limiting, that stem cell proliferation serves as the mechanistic explanation for the observed improvements in behavioral and histological observations for induced stroke animals compared to the control group. Thus, further improvement in stimulation of stem cell proliferation by adding Spirulina and/or AFA-Omega (EtOH), as evidenced by in vitro study, is likely to improve behavioral performance and histological results in vivo.

Sanberg et al. also provides evidence of migration of newly proliferated stem cells to a site of injury and provides the strongest possible evidence for the efficacy of Composition A in stimulating stem cell proliferation as the mechanism responsible for protection from induced-stroke damage. Thus, a method of protecting from damage caused by a stroke includes selecting a supplement that substantially increases (i.e. at least 10%) the proliferation of stem cells responsible for repairing damaged brain tissues, such a neuronal tissues, as compared to a control, and administering the supplement, such as Composition A and/or Spirulina and/or AFA-Omega (EtOH) in a therapeutic dose, such as by ingestion, injection or the like. The therapeutic dose of Composition A may be decreased by the addition of Spirulina and/or AFA-Omega (EtOH). A similar method may be used for preventing or repairing damage due to heart disease, if the compound selected is selected to stimulate proliferation of stem cells capable of repairing damage to the heart muscle or other cardio-arterial tissues. A similar method may be used for preventing or repairing damage to pancreatic stem cells, bone marrow stem cells, and any other differentiated stem cells.

A compound comprised of an extract or preparation of blueberries and/or Spirulina and/or AFA-Omega (EtOH) may be compounded with at least one of a group of substances, synergistically, the group consisting of carnosine, catechin, vitamin D₃, and green tea extract, may be formed and tested in vitro to determine efficacy for use in stimulating proliferation of any of the variety of adult stem cells. FIGS. 2, 1B, 3B, 4B and 5B show that compositions may have more or less efficacy in substantially stimulating proliferation of one adult stem cell than another, but the proliferation of all three types of stem cells tested was substantially increased, synergistically, by combinations of some combination of these substances. Furthermore, the effectiveness of a combined compound in vitro has been shown to predict effective protection and/or repair in vivo.

Based on this disclosure, preparing therapeutic compounds that are effective in stimulating a substantial increase in a particular type of stem cell proliferation is within the skill of a person of ordinary skill in the field of supplement compounding, without undue experimentation, for the following substances: blueberry (extract and other preparations), the dietary supplement known as Spirulina, AFA-Omega (EtOH-prepared), vitamin D₃, carnosine, catechin, green tea (extract and other preparations), and derivatives and combinations thereof. In one method, a compound is identified for substantially increasing proliferation of a particular variety of stem cell, by screening in an in vitro experimental design, Compound A, Compound A with Spirulina and/or AFA-Omega (EtOH), a compound of blueberry, catechin, and Spirulina and/or AFA-Omega (EtOH), and a compound of blueberry, vitamin D3, carnosine, and Spirulina and/or AFA-Omega (EtOH). Results of the experimental design may be used to add or remove one or more of the substances from a compound designed to substantially increase proliferation of the particular stem cell tested compared to a non-optimized compound, such as Composition A, which appears to stimulate a substantial increase in all stem cell lines tested. By such a design, an optimized compound may be selected that preferentially stimulates a substantially greater (i.e. at least 10%) proliferation of one or a select group of stem cells compared to its effect on proliferation of other stem cells.

For example, Composition A leads to increased expression of neuronal phenotypes, bolstering the evidence of a mechanism of neurogenesis underlying Composition A neuro-protection. Composition A enhances neuronal differentiation of newly formed cells in the ischemic striatum, Composition A supplementation does not enhance differentiation of newly formed cells into glial lineage in the ischemic striatum. Instead, Composition A induces neural differentiation, with increased tendency towards neuronal over glial lineage, for example. This is preferred. Composition A with addition of Spirulina and/or AFA-Omega (EtOH) is used to further stimulate proliferation of neuronal stem cells, for example. Pathological manifestation of stroke, at least in this MCAo model, is characterized by extensive neuronal loss accompanied by increased glial cell activation. Thus, it is thought that an addition of an amount of Spirulina and/or AFA-Omega (EtOH) that is capable of stimulating both the proliferation of stem cells and/or differentiation of stem cells into neuronal cells is beneficial to prevent further damage and/or help to repair damage caused by stroke. A robust neuronal differentiation at two weeks post-stroke is equally advantageous since a rapid cell death cascade proceeds after the stroke onset. The preferential neuronal differentiation during the acute stroke phase, as disclosed in Sanberg et al., provides a solid evidence that neurogenesis is capable of playing a major active role in mediated neuro-protection. Thus, it is believed that there are multiple advantages to using supplementation effective in repairing damage caused by injuries such as stroke in mammals.

Compounds, such as Compound A and other compounds formed using disclosed substances, may be added to cell cultures as described in the results section for in vitro assay, for example.

Examples of sources of compounds for Composition A are as follows: blueberry (freeze dried powder, Van Drunen Farms, Momence, Ill. and/or Vitablue and/or extracts of blueberries and/or other preparations), green tea extract (Rexall), carnosine (Sigma) and the activated form of vitamin D3 (25-Hydroxy-cholecalciferol). In addition, the dietary supplement known as Spirulina from Earthrise Nutritionals may be added, as described herein, for example. Composition A comprises 500 ng/ml blueberry, 20 μM carnosine, 500 ng/ml green tea, and 5 μM vitamin D3, for example. Spirulina may be prepared in a manner similar to the compounding of the substances in Composition A for adding to Composition A and forming a new compound. For example, Spirulina may be dissolved in distilled water, sonicated for 15 minutes and filter sterilized with a 45 micrometer filter prior to application to cell cultures in vitro. This form of Spirulina is referred to herein as a suspension. The source of Spirulina for use in preparing the suspension may be a dried powder. For example, the dried powder is dried using a laboratory or industrial spray dryer. In one example, care is taken during the drying process to reduce or prevent oxidation, preserving antioxidant properties of the dried powder, as well as any oxygen sensitive vitamins, enzymes, or phytonutrients. For example, Spirulina may be dried in step using an oxygen getter, a protective atmosphere, or after a step of pulling a vacuum prior to introducing a protective atmosphere or an oxygen getter. The protective atmosphere may use an inert gas or other protective gas, such as nitrogen, argon or carbon dioxide. The temperature during drying may be regulated to prevent heating of the Spirulina above a temperature of 41 degrees centigrade. For example, the temperature regulation may heat the Spirulina above the body temperature of a human for less than two minutes in order to prevent oxidation and/or other chemical reactions associated with heating. Alternatively, Spirulina may be processed as an ethanol extract. Ethanol evaporates at lower temperatures and more quickly than water, for example. Other processes may be used to suspend or extract Spirulina for compounding, also. So long as oxidation and temperature is controlled, the process of compounding Spirulina is not known to affect its potency in stimulating proliferation of stem cells.

Cell cultures and MTT Assay. As an example of cell proliferation assay, human bone marrow cells and human CD34⁺ cells (Cambrex, Inc.) were cultured in 96 well plates (5×10⁴/well) containing 100 μL of complete medium (RPMI 1640 medium supplemented with 5% fetal calf serum). These cells were treated for 72 hours with various substances/compounds using a range of doses (15 ng/mL to 500 ng/mL or 0.3125 μM to 20 μM), where ng is nanograms, mL is milliliters and μM is micromoles. Five hours before the end of the treatment, 20 μL of MTT solution (MTT kit, Sigma) was added to each well. These plates were then incubated in a CO₂ incubator at 37° C. for 5 hours and the cultured media removed with needle and syringe. 200 μL of DMSO was added to each well with pipetting up and down to dissolve crystals. These plates were put back into the 37° C. incubator for 5 minutes, transferred to plate reader and measured absorbance at 550 nM. Data were represented as relative percentage mean proliferation, defined as O.D. reading number of each treated cells normalized to control cells (in the absence of treatment). The percent over control is defined as percent values that exceed MTT results from baseline conditions of cell culture grown in RPMI medium plus 5% fetal calf serum. The percent over control values are reported in the drawings.

Human Bone Marrow cells. FIG. 1A shows a dose response using Spirulina alone on proliferation of bone marrow cells as measured by the MTT assay. At 125 ng/ml, Spirulina showed greater than 10% increase in bone marrow cell proliferation, which is considered a substantial increase. Any increase in proliferation less than 10% is not considered a substantial increase in proliferation of stem cells and is considered a failed assay that does not warrant further testing of the substance at the does tested.

FIG. 1A shows that lower doses do not show any substantial increase, and 125 ng/ml just provides enough stimulation to achieve a substantial increase in proliferation. The increase in proliferation appears to be nearly linear with dose, although doubling the dose from 32.25 to 62.5 to 125 does not quite achieve a corresponding doubling of stem cell proliferation.

Thus, the investigators were very surprised to see a marked synergistic effect upon addition of 125 ng/ml of Spirulina to Compound A (labeled NT020), as shown in FIG. 1B. As disclosed in Sanberg et al., Spirulina had been discounted as a constituent of a compound due to an initial assay showing low activity for stem cell proliferation. Nevertheless, testing of Spirulina as an additive to Compound A evidenced a synergistic effect, yielding a substantial increase compared to Compound A, alone. This result is even more surprising considering that AFA suspensions using water showed no substantial increase in proliferative effect over Compound A, alone, when added to Compound A during in vitro assay. It was very unexpected that Spirulina at 125 ng/ml, when added to Compound A, nearly doubled the effect of bone marrow cell proliferation over Compound A, alone.

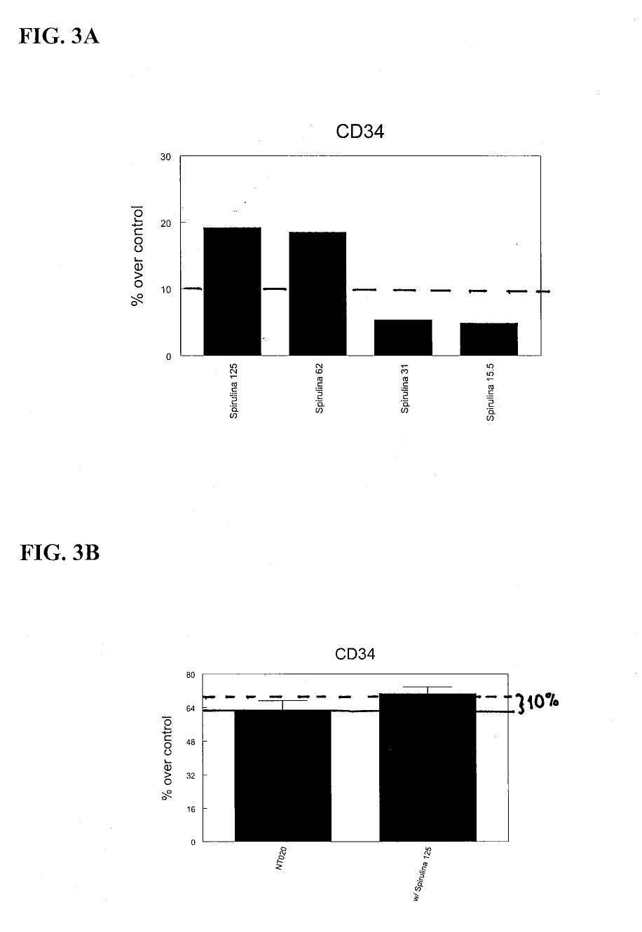

CD34+ stem cells. FIGS. 3A and 3B show graphs of an assay for demonstrating the effectiveness of Spirulina for enhancing proliferation of CD34+ cells. Surprisingly, Spirulina at dosages of 62 ng/ml and 125 ng/ml was associated with an increase in proliferation of CD34+ stem cells of nearly 20%. The results at a dosage of 62 ng/ml were substantially better than the results using the same dose for human bone marrow stem cells. In human bone marrow stem cells the effect on proliferation was nearly linear, but the effect on CD34+ indicates that there is a critical threshold for observing a substantial increase in CD34+ proliferation. This is a surprising and unexpected result. Even though Spirulina at a dose of 62.5 ng/ml shows almost a 20% increase in the CD34+ proliferation assay, the results of combining Spirulina with Compound A barely achieved a level of proliferation of 10% over the control. This result for CD34+ is more consistent with results observed with other blue green algae (except AFA-Omega (EtOH)), which demonstrated stem cell proliferation associated with much less than an additive effect over the results of Compound A, alone. Thus, the observations of a synergistic effect of a combination of Spirulina and Compound A is even more surprising and unexpected. From the data presented, it is apparent that only some, but not all, varieties of stem cells benefit synergistically from a combination of Spirulina and Compound A.

AFA-Alpha & AFA-Omega. Aphanizomenon flos-aqaue (AFA) was prepared from products of Simplexity Health known as Alpha Sun® (AFA-ALPHA) and Omega Suns (AFA-OMEGA).¹ AFA is collected from upper Klamath Lake, is cleaned, is dewatered and is screened on a lake harvester. Then, the AFA is cleaned a second time in land processing facilities, is frozen and is stored prior to drying.
¹Alpha Sun® or Omega Sun®, are registered trademarks of Simplexity Health.

AFA-OMEGA. AFA-OMEGA is distinguished from AFA-ALPHA due to processing after harvesting of the AFA. AFA-OMEGA is put through a process that removes the cell wall of the cyanobacteria. Feed materials may be fresh, raw processed, or thawed frozen Aphanizomenon flos-aquae, for example. Thus, AFA-OMEGA is AFA-ALPHA after further processing to remove the cell walls. After the second cleaning step, AFA is pumped from a feed tank into a homogenizer which applies 3000 psi pressure to the AFA cells, which ruptures its cell wall. For example, the homogenizer is an integrated design of 3-5 cylinders, pump valves, plunger lubrication and inlet/outlet in a single block. One example is capable of inducing a pressure in a range of up to 600 bar (8700 psi). Until the AFA with ruptured cell walls (homogenized) is pumped into a centrifuge, it may be stored in a jacketed cooling tank. Homogenized AFA is pumped into a separator centrifuge, which separates cell wall material from intracellular material via centrifugal force. Examples used in industrial separation processes include nozzle separators and self-desludging centrifuges, but any separator may be used to separate the cell wall and intracellular materials. The intracellular material is AFA-OMEGA, which may be frozen and stored prior to drying into a powder form, for example.

Water Suspension. AFA products may be obtained in a powdered form, for example. Powdered AFA may be hydrated to make an AFA suspension/solution. For example, dried AFA is suspended/dissolved in distilled water, sonicated for 15 minutes and filter sterilized with a 45 micrometer filter prior to use in a proliferation assay. This is referred to herein as water suspension of AFA, which is also represented by AFA being associated with H2O or without further labeling. Water suspension of AFA is the default preparation route, and it was used to prepare both AFA-Alpha and AFA-Omega.

Spray Drying of AFA. A preferred drying process to produce AFA powder is spray drying. Spray drying may be modified to prevent oxidation in order to preserve natural vitamins, enzymes, and phytonutrients, as previously described. Also, as previously described for Spirulina, the temperature of the spray drier may be controlled to reduce or prevent chemical changes to the AFA.

AFA (EtOH). AFA preparations may be prepared as ethanol extracts. AFA may be dissolved/suspended in 70% ethanol, vortexed for 40 seconds and incubated at 65° C. for 2 hours. Ethanol extracts may centrifuged at 1000 RPM for 2 minutes. Then, the supernatant may be collected. This process may be repeated (one or more times). Then, the supernatants may be combined and dried. AFA that is denoted as (EtOH) or ethanol-processed herein has undergone this process. The ethanol extract of AFA may be weighed, resuspended in water, and filter sterilized. If dried, then AFA (EtOH) is first resuspended in water prior to use in stem cell proliferation assays.

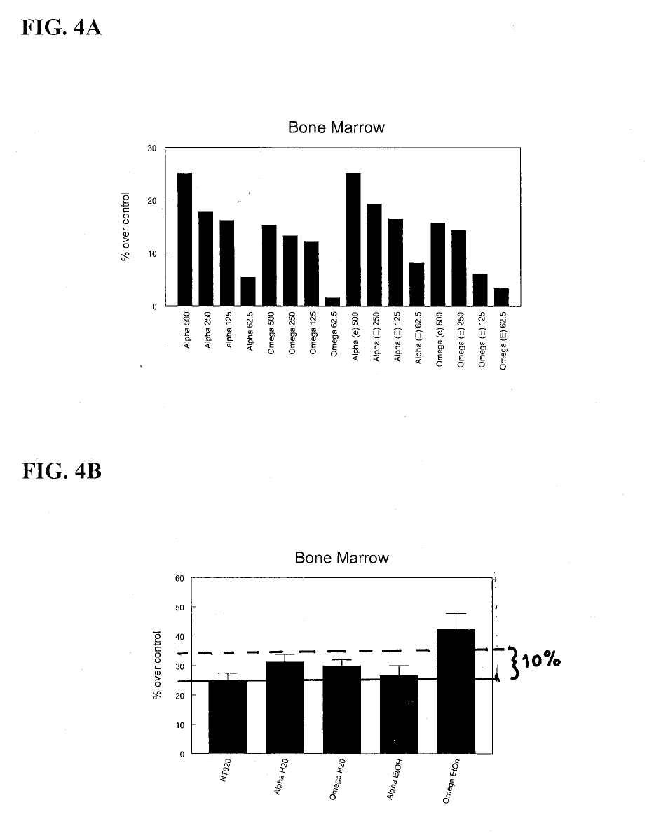

FIG. 4A shows a dose response of AFA-Alpha (Alpha) and AFA-Gamma (Gamma) either processes as a water suspension (no additional label) or as an ethanol-processed extract (labeled e or E). Thus, Alpha, Alpha (E), Omega and Omega (E) were assayed for effect on proliferation of bone marrow stem cells. Also, each of these four were assayed at four concentrations: 62.5, 125, 250 and 500 ng/ml (i.e. doubling the previous dose). From the data in FIG. 4A, it is apparent that doubling of the dose from 250 to 500 ng/ml is subject to diminishing returns, because doubling the amount of the dose fails to come anywhere close to doubling of the proliferation of stem cells. Indeed, even the increase in proliferation due to doubling from 125 to 250 ng/ml fails to come anywhere close to being doubled, except for Omega (E) 125 to Omega (E) 250, which appears to more than double the % proliferation over the control in an MTT assay. The Alpha water suspension and Alpha ethanol extracts exhibited better proliferative effects than either of the Omega forms of AFA.

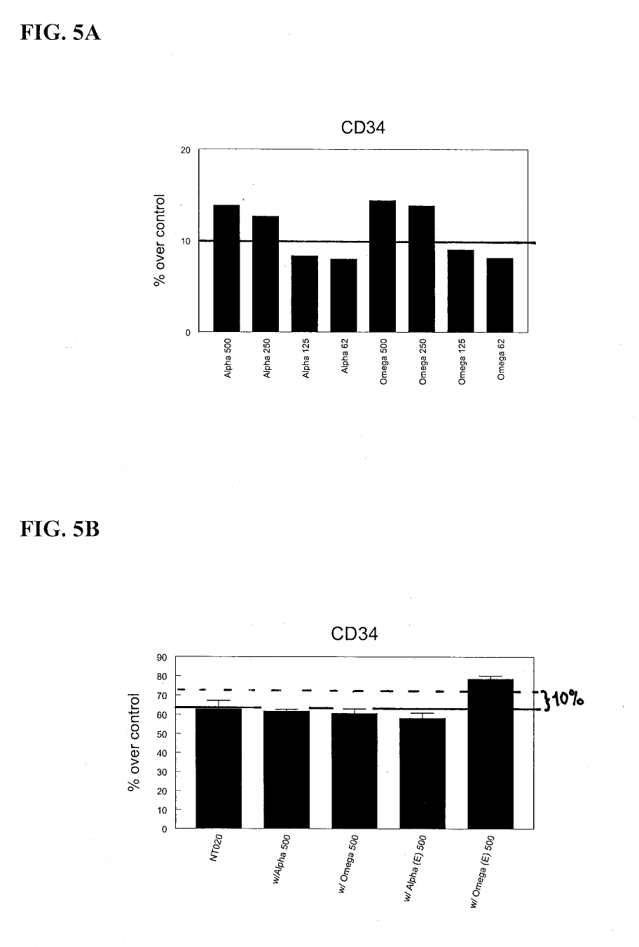

FIG. 4B reports results of the MTT assay when the AFA tested in FIG. 4A was added at a concentration of 500 ng/ml to Composition A (labeled NT020). FIG. 4B may be used to compare the effectiveness of Composition A, alone, to the combination of Composition A and AFA-Alpha or AFA-Omega. Strikingly, while all of the AFA substances tested in FIG. 4A showed a substantial increase in proliferation over control at a concentration of 500 ng/ml, only AFA-Omega (EtOH) presented a substantial increase in proliferation over Composition A, alone, when combined with Composition A. A comparison of FIGS. 5A and 5B, which report the MTT assay for CD34+ cells, is even more striking. Both AFA-Alpha (water suspension) and AFA-Omega (water suspension) exhibited a substantial increase in proliferation over control, in the absence of Composition A. However, all of the AFA substances, except for AFA-Omega (EtOH) exhibited a reduction or no change in proliferation, when combined with Composition A, compared to Composition A, alone. Suprisingly, in contrast to this trend, the MTT assay for AFA-Omega (EtOH) at a concentration of 500 ng/ml, in combination with Composition A, exhibits substantially increased proliferation compared to Composition A, alone. In contrast, AFA-Alpha (EtOH), which was subjected to the same ethanol extraction process, was the worst performing AFA substance in combination with Composition A. Without limiting the invention in any way, it is thought that ethanol extraction of AFA-Omega, which has had its cell walls removed, might concentrate one or more substances that synergistically stimulate CD34+ proliferation in combination with Composition A. While the active ingredient that is present in AFA-Omega (EtOH) is not known, the product produced by this process has some active ingredient or ingredients that are, perhaps, not present or are present in much lower concentration in the AFA substances prepared by alternative processing methods. It is clearly demonstrated that the active ingredient of AFA-Omega (EtOH) substantially increases stem cell proliferation for CD34+ cells and bone marrow stem cells compared to Composition A, alone.

As there was no difference between the ethanol extract of AFA brands and the water suspensions of the AFA in the MTT assay for bone marrow stem cell proliferation, only the normal water suspensions were studied. At dosages of 250 ng/ml and 500 ng/ml, both water suspensions of AFA-Omega and AFA-Alpha exhibited substantial increases (i.e. 10%) in CD34+ stem cell proliferation over control. There appeared to be a critical dose between 125 ng/ml and 250 ng/ml for achieving at least 10% increased proliferation. Doubling the dose to 500 ng/ml was not associated with much of an increase in proliferation. The data shows that doubling the dose from 62.5 to 125 ng/ml also failed to produce much of an increase in proliferation.

FIG. 19 demonstrates the effects of the AFA algae when used in conjunction with the proprietary formulation of NT020. As was observed with the bone marrow cells, although all individual compounds showed some activity alone, only the Omega Sun® ethanol extract (in this example, 500 ng/ml) demonstrated additional activity (i.e., 10% or more additional activity over NT020 with respect to stem cell proliferation as measured by the MTT assay.

Many other combinations and doses will be apparent to an artisan based on the examples and ranges provided for combinations of ingredients. Some synergistic effects are shown for combinations of two or more of the listed active ingredients. Ranges of effective doses may be tailored for a specific mammal by comparing the amount of each substance measured in blood serum levels, as compared to a known animal or human blood serum levels.

What is claimed is:

1. A method of increasing stem cell proliferation comprising:
preparing blueberry or a blueberry extract for compounding;
preparing AFA-OMEGA (EtOH) for compounding;
selecting and preparing a substance from the group of substances consisting of carnosine, catechin, green tea extract, vitamin D3, and derivatives and combinations thereof for compounding;
compounding the blueberry or a blueberry extract, the AFA-OMEGA (EtOH), and the substance selected in the step of selecting, forming a compound; and
administering the compound in a therapeutically effective dose.

2. The method of claim 1, wherein the step of selecting is further limited to selecting vitamin D3.

3. The method of claim 2, wherein the step of selecting is further limited to selecting green tea extract.

4. The method of claim 1, wherein the step of selecting includes selecting carnosine, catechin, or a derivative or combination of carnosine or catechin.

5. The method of claim 1, wherein the step of selecting is further limited to selecting carnosine, green tea extract, and vitamin D3.

6. The method of claim 1, wherein the step of selecting is further limited to selecting a substance capable of synergistically stimulating a substantial increase in proliferation of phenotypes useful for repairing cells damaged by injury and disease.

7. The method of claim 6, wherein the step of administering includes diagnosing a brain injury capable of benefiting from the step of administering and selecting a dose and schedule for administering of the dose capable of substantially increasing expression of neuronal cells following brain injury.

8. The method of claim 1, further comprising preparing a dietary supplement known as Spirulina for compounding prior to the step of compounding, the step of compounding including the dietary supplement known as Spirulina.

9. A composition comprising:
blueberry or a derivative thereof;
a AFA-OMEGA (EtOH); and
a substance selected from the group of substances consisting of carnosine, green tea extract, catechin, and vitamin D3 or derivatives, combinations, or derivatives and combinations thereof.

10. The composition of claim 9, wherein the substance includes vitamin D3.

11. The composition of claim 10, wherein at least 50 mg vitamin D3 is present in the composition.

12. The composition of claim 11, wherein the composition comprises at least 400 milligrams of blueberry.

13. The composition of claim 12, wherein the composition includes green tea extract in a range from 400 mg to 5 grams.

14. The composition of claim 13, further comprising: at least 400 mg of green tea and at least 10 mg of carnosine.

15. The composition of claim 9, further comprising a dietary supplement known as Spirulina.

16. A system for preventing and repairing cell damage in a animal caused by aging, injury, or disease, comprising:
stimulating a substantial increase in stem cell proliferation by administering a therapeutically effective amount of a combination of blueberry or a derivative thereof, a AFA-OMEGA (EtOH) or a derivative thereof, and at least one substance or derivative or extract of the substance selected from the group of substances consisting of carosine, catechin, green tea, vitamin D3, and combinations thereof; and
continuing the step of administering for at least an effective duration.

17. The system of claim 16, wherein the substance includes vitamin D3 or a derivative thereof.

18. The system of claim 17, wherein the substance includes green tea extract.

19. The system of claim 18, wherein the substance includes carnosine.

20. The system of claim 19 wherein the administering includes at least 400 mg of blueberry or derivative thereof, at least 400 mg of green tea extract, and at least 50 mg of vitamin D3 or a derivative thereof in a compound administered at least once daily.

21. The system of claim 19, wherein the administering includes a range of 400 mg to 5 grams of blueberry or a derivative thereof, 400 mg to 5 grams of green tea extract, 2000 IU to 4000 IU of the vitamin D3 or a derivative thereof and 10 mg to 100 mg of carnosine in a compound administered at least once daily.
